(19) United States
(12) Patent Application Publication
(10) Pub. No.: US 2015/0368632 A1
(43) Pub. Date: Dec. 24, 2015

(54) CRYSTALLINE MATERIALS ON BIOLOGICAL TISSUE AND METHODS FOR MAKING THE SAME

(71) Applicant: VANDERBILT UNIVERSITY, Nashville, TN (US)

(72) Inventors: Charles M. LUKEHART, Nashville, TN (US); Borislav L. IVANOV, Nashville, TN (US); Jeremiah C. BEAM, Nashville, TN (US); Matthew S. WELLONS, Aiken, SC (US); Andrew G. HARRIS, Antioch, TN (US)

(21) Appl. No.: 14/766,535

(22) PCT Filed: Feb. 7, 2014
(86) PCT No.: PCT/US2014/015339

§ 371 Date: Aug. 7, 2015

Related U.S. Application Data
(60) Provisional application No. 61/762,567, filed on Feb. 8, 2013.

(51) Int. Cl. C12N 13/00; C12N 5/04; C12N 5/077

(52) U.S. Cl. CPC C12N 13/00; C12N 5/0654; C12N 5/04; C12N 2529/10; C12N 2533/00

(57) ABSTRACT

Provided are compositions including a direct interface between a biological tissue and a crystalline material, wherein the crystalline material has a crystallization temperature that exceeds the temperature at which the biological tissue incurs thermal damage. Also provided are methods for producing said compositions. The ability to apply crystalline materials to biological tissues has wide applicability, e.g., with cyborg applications. Extensive challenges exist, however, in applying crystalline materials to biological tissue substrates.

### CROSS-REFERENCE TO RELATED APPLICATIONS

This patent application claims priority to U.S. Provisional Patent Application No. 61/762,567, filed Feb. 8, 2013, the content of which is incorporated herein by reference in its entirety.

### GOVERNMENT LICENSE RIGHTS

This invention was made with government support under contract nos. EPS-1004083 and DGE 0909667 awarded by National Science Foundation. The government has certain rights in this invention.

### BACKGROUND

The ability to apply crystalline materials to biological tissues has wide applicability, e.g., with cyborg applications. Extensive challenges exist, however, in applying crystalline materials to biological tissue substrates. For example, limitations exist with vapor deposition techniques that require a vacuum or high-temperature equipment. Additionally, biological tissues typically thermally decompose at high temperatures.

Accordingly, there is a need for new compositions and methods.

### SUMMARY

In one embodiment, the invention provides a composition comprising a crystalline material attached directly to a biological tissue.

In another embodiment, the invention provides a composition comprising a crystalline material formed directly on the surface of a biological tissue.

In another embodiment, the invention provides a composition comprising a direct interface between a biological tissue and a crystalline material, wherein the crystalline material has a crystallization temperature that exceeds the temperature at which the biological tissue incurs thermal damage.

In another embodiment, the invention provides a method of producing a modified biological tissue, the method comprising illuminating a biological tissue and a material precursor with pulses of light of sufficient duration and intensity to generate a crystalline material on the biological tissue without damaging the biological tissue.

In another embodiment, the invention provides a method of directly attaching a crystalline material to a biological tissue, the method comprising illuminating a biological tissue and material precursor with pulses of light in the absence of air to generate a plasma and form a crystalline material directly attached to the biological tissue without damaging the biological tissue.

In another embodiment, the invention provides a method of forming a crystalline material directly on the surface of a biological tissue, the method comprising: converting a material precursor into a crystalline material within a plasma generated by pulses of light in a spatially confined area; and forming a modified biological tissue having the crystalline material thereon.

Other aspects of the invention will become apparent by consideration of the detailed description and accompanying drawings.

### BRIEF DESCRIPTION OF THE DRAWINGS

FIG. 1 is a scanning electron microscopy image of ReB₂ microcrystals on green onion tissue produced by Confined-Plume Crystal Deposition (CPCD) using an 800-nm laser showing artificial brightness enhancement of one onion cell wall.

FIG. 2 is a scanning electron microscopy image of ReB₂ platelets on green onion tissue produced by CPCD using an 800-nm laser.

FIG. 3 is an x-ray diffraction spectroscopy spectrum of ReB₂ on green onion tissue produced by CPCD using an 800-nm laser. The line pattern is of standard bulk, crystalline ReB₂.

FIG. 4 is an energy-dispersive x-ray spectroscopy spectrum of ReB₂ on green onion tissue produced by CPCD using an 800-nm laser.

FIG. 5 is a scanning electron microscopy image of CdS microcrystals on green onion tissue produced by CPCD using an 800-nm laser at lower magnification.

FIG. 6 is a scanning electron microscopy image of CdS microcrystals on green onion tissue produced by CPCD using an 800-nm laser at higher magnification.

FIG. 7 is an x-ray diffraction spectroscopy spectrum of CdS microcrystals on green onion tissue produced by CPCD using an 800-nm laser. The line pattern is of standard bulk, greenockite (wurtzite CdS).

FIG. 8 is a scanning electron microscopy image of CdS microcrystals on green onion tissue produced by CPCD using a 2.94-μm laser at lower magnification.

FIG. 9 is a scanning electron microscopy image of CdS microcrystals on green onion tissue produced by CPCD using a 2.94-μm laser at higher magnification.

FIG. 10 is an x-ray diffraction spectroscopy spectrum of CdS microcrystals on green onion tissue produced by CPCD using a 2.94-μm laser. The line pattern is of standard, bulk greenockite (wurtzite CdS).

FIG. 11 is a scanning electron microscopy image of Au microcrystals on green onion tissue produced by CPCD using an 800-nm laser at lower magnification.

FIG. 12 is a scanning electron microscopy image of Au microcrystals on green onion tissue produced by CPCD using an 800-nm laser at higher magnification.

FIG. 13 is an x-ray diffraction spectroscopy spectrum of Au microcrystals on green onion tissue produced by CPCD using an 800-nm laser. The line pattern is of standard, bulk greenockite (wurtzite CdS).

FIG. 14 is a scanning electron microscopy image of ReB₂ platelets on green onion tissue produced by CPCD using an 800-nm laser at lower magnification.

FIG. 15 is a scanning electron microscopy image of ReB₂ platelets on green onion tissue produced by CPCD using an 800-nm laser at higher magnification.

FIG. 16 is an x-ray diffraction spectroscopy spectrum of ReB₂ platelets on green onion tissue produced by CPCD using an 800-nm laser. The line pattern is of standard, bulk ReB₂.

FIG. 17 is a scanning electron microscopy image of ReB₂ microcrystals on cow femur tissue produced by CPCD using a 535-nm laser at lower magnification.

FIG. 18 is a scanning electron microscopy image of ReB₂ microcrystals on cow femur tissue produced by CPCD using a 535-nm laser at higher magnification.

FIG. 19 is an x-ray diffraction spectroscopy spectrum of ReB₂ platelets on cow femur tissue produced by CPCD using a 535-nm laser. The solid line pattern is of standard, bulk ReB₂ and the dashed line pattern is of hydroxyapatite structure.

### DETAILED DESCRIPTION

Before any embodiments of the disclosure are explained in detail, it is to be understood that the disclosure is not limited in its application to the details of construction and the arrangement of components set forth in the following description or illustrated in the following drawings. The disclosure is capable of other embodiments and of being practiced or of being carried out in various ways. Other aspects of the disclosure will become apparent by consideration of the detailed description and accompanying drawings.

It is specifically understood that any numerical value recited herein (e.g., ranges) includes all values from the lower value to the upper value, i.e., all possible combinations of numerical values between the lowest value and the highest value enumerated are to be considered to be expressly stated in this application. For example, if a concentration range is stated as 1% to 50%, it is intended that values such as 2% to 40%, 10% to 30%, or 1% to 3%, etc., are expressly enumerated in this specification. These are only examples of what is specifically intended. With respect to amounts of components, all percentages are by weight, unless explicitly indicated otherwise.

The present disclosure generally relates to deposition of crystalline materials onto biological tissue. Confined-plume chemical deposition (CPCD) is a process for preparing microcrystalline coatings of compositions on a variety of materials. Previously, crystalline materials were deposited on the surface of Si, NaCl, Kevlar, Teflon, and various polymers using CPCD. See Ivanov et al., “Confined-Plume Chemical Deposition: Rapid Synthesis of Crystalline Coating of Known Hard or Superhard Materials on Inorganic or Organic Supports by Resonant IR Decomposition of Molecular Precursors,” J. Am. Chem. Soc. 2009, 131, 11744-50, which is incorporated herein in its entirety by reference.

The present compositions and methods generate crystalline material directly on biological tissue. In one embodiment, present disclosure provides for deposition of crystalline materials onto biological tissue by irradiating molecular precursor/substrate assemblies confined between two transparent windows with femtosecond laser pulses which generate a spatially and temporally confined plasma within the precursor layer leading spontaneously to nucleated growth and deposition of microcrystalline coating on any chosen hard or soft substrate with no observed thermal damage to the substrate.

Examples of biological tissue include, without limitation, at least one of animal kingdom biological tissue, plant kingdom biological tissue, bacterial kingdom biological tissue, fungal kingdom biological tissue, archaeal kingdom biological tissue, protistal kingdom biological tissue, and combinations thereof. Examples of animal kingdom biological tissue include, without limitation, at least one of mammalian tissue, epithelial tissue, bone tissue, muscle tissue, and nervous tissue, and combinations thereof. Examples of plant kingdom biological tissue include, without limitation, at least one of onion, bacteria, parenchyma tissue, collenchyma tissue, sclerenchyma tissue, xylem tissue, phloem tissue, and epidermis tissue, and combinations thereof.

Examples of crystalline materials include, without limitation, at least one of crystalline ceramic materials, crystalline metal materials, semiconductor materials, piezoelectric materials, ceramic bone mimics, and combinations thereof.

Examples of crystalline ceramic materials include, without limitation, at least one of metal oxide, metal carbide, metal boride, metal nitride, mixed-metal oxide, mixed-metal carbide, mixed-metal boride, mixed-metal nitride, elemental boride, elemental carbide, elemental nitride and combinations thereof. Particularly suitable crystalline ceramic materials are ReB₂, RuB₂, WB₂, and B₄C. Examples of crystalline metal materials include, without limitation, at least one of Au, Pt, Ag, Pd, Pb, Sn, Bi, Sb, Te, transition metal, lanthanide metal and combinations thereof. Examples of semiconductor materials include, without limitation, at least one of C, Si, Ge, any II-VI semiconductor, AB, where A=Be, Mg, Zn, Cd, or Hg and B═O, S, Se, or Te, any III-V semiconductor, AB, where A=B, Al, Ga, or In and B═N, P, As, or Sb, any IV-VI semiconductor, AB, where A=Ge, Sn, or Pb and B═O, S, Se, or Te, and combinations thereof. Examples of piezoelectric materials include, without limitation, at least one of ZnS, ZnO, PbZrO₃, PbTiO₃, or Pb(Zr,Ti)O₃, and combinations thereof. Examples of ceramic bone mimics include, without limitation, at least one of hydroxyapatite, beta-tricalcium phosphate, silica, or metal phosphate, and combinations thereof.

The crystalline materials are formed from a variety of material precursors that are exposed to a suitable light source. Examples of ceramic material precursors include, without limitation, at least one of any compound containing both the desired metal or element and oxygen, carbon, boron, silicon, aluminum or nitrogen, any compound containing the desired metal or element, any ceramic compound containing oxygen, carbon, boron, silicon, aluminum or nitrogen having stoichiometries conducive for ceramic formation, and combinations thereof. Particularly suitable ceramic material precursors are (B₃H₈)Re(CO)₄, Ru₃(CO)₁₂/B₁₀H₁₄, W(CO)₆/B₁₀H₁₄, and o-B₁₀C₂H₁₂. Examples of metal material precursors include, without limitation, at least one of any compound containing the desired metal or metals, and combinations thereof. Examples of semiconductor material precursors include, without limitation, at least one of any compound containing both the desired “A” and “B” elements, any compound containing the desired “A” element, any compound containing the desired “B” element, and combinations thereof. Examples of piezoelectric material precursors include, without limitation, at least one of any compound containing both Zn, Pb, Zr, or Ti and S or O, any compound containing Zn, Pb, Zr, or Ti, any compound containing S or O, and combinations thereof. Examples of ceramic bone mimic precursors include, without limitation, at least one of any compound containing both Ca, Si, or other metal and P or O or phosphate, and combinations thereof.

Suitable light sources include any light source capable of producing pulses of light exceeding a minimal threshold of intensity and duration to generate a plasma, but not exceeding a maximal threshold of intensity and duration that would result in damage to the polymer substrate. Examples of suitable light sources include, without limitation, at least one of femtosecond near-IR lasers such as Ho:YAG, Tm:YAG, Yb:YAG, Ti:sapphire laser, picosecond near-IR lasers such as Ho:YAG, Tm:YAG, Yb:YAG, Nd:YAG, Nd:YLF lasers, nanosecond near-IR lasers such as Er:YAG, Er,Cr:YSGG, Ho:YAG, Tm:YAG, Yb:YAG, Nd:YAG, Nd:YLF lasers, femtosecond, picosecond and nanosecond Optical Parametric Oscillator tunable IR, near-IR, and visible lasers, picosecond and nanosecond visible lasers such as ruby, alexandrite, diode lasers, nanosecond visible lasers such as Cu and Au metal vapor lasers, microsecond IR and near-IR lasers such as Er:YAG, Er,Cr:YSGG, Ho:YAG, Tm:YAG, Yb:YAG, Nd:YAG, Nd:YLF lasers, UV excimer laser such as XeCl, KrF, ArF, and F₂ lasers, and Second Harmonic Generation of Nd:YAG, Nd:YLF and Ti:sapphire laser and combinations thereof. One particularly suitable light source is an amplified Ti:sapphire laser. Another particularly suitable light source is a 2.94-μm microsecond Er:YAG laser.

Pulses of light may be of any duration sufficient to generate a plasma, but short enough to avoid damaging the biological tissue. Pulses of light may be between about 1 fs and about 300 μs (full width at half maximum “FWHM”) in duration. Pulses of light may be of duration shorter than about 300 μs (FWHM), shorter than about 1 ns (FWHM), shorter than about 1 ps (FWHM), or shorter than about 100 fs (FWHM). Pulses of light may be of duration longer than about 1 fs (FWHM), longer than about 1 ps (FWHM), longer than about 1 ns (FWHM), or longer than about 100 ns (FWHM). Pulses of light may be about 150 fs (FWHM) in duration.

Pulses of light may be of any energy that is sufficient to generate a plasma, but also low enough to avoid damaging the biological tissue. Pulses of light may have energy from about 10 nJ to about 1 J. Pulses of light may have energy less than about 1 μJ, less than about 1 mJ, less than about 1 J. Pulses of light may have energy more than about 1 mJ, more than about 1 μJ, or more than about 10 nJ. Pulses of light may have energy of about 1 mJ.

Pulses of light may be of any wavelength capable of generating a plasma. Pulses of light may have a wavelength centered at from about 0.4 μm to about 11 μm. Pulses of light may have a wavelength centered at shorter than about 11 μm shorter than about 1 μm. Pulses of light may have a wavelength centered at longer than about longer than about 1 μm, or longer than about 0.4 μm. Pulses of light may have a wavelength centered at about 800 nm.

Pulses of light are focused to a spot size suitable for generating a plasma. Pulses of light may be focused to a spot size from about 1 μm to about 5 cm by about 1 μm to about 5 cm. Pulses of light may be focused to a spot size of less than about 1 cm or less than about 1 mm by less than about 1 cm or less than about 1 mm. Pulses of light may be focused to a spot size of more than about 1 μm or more than about 1 mm by more than about 1 μm or more than about 1 mm. Pulses of light may be focused to a spot size of 0.1 mm×6 mm.

The plasma is generated by illuminating the biological tissue and precursor with the focused pulses of light in a spatially confined area. A spatially confined area is an area isolated from the atmosphere. Isolated from the atmosphere means situated such that the precursor cannot be vaporized into the atmosphere and also cannot react with the atmospheric gases.

Suitable materials for confining the precursor in a spatially confined area include, without limitation, at least one of sapphire wafers, quartz wafers, glass wafers, any crystalline or amorphous materials transparent for the laser wavelength used, and combinations thereof. Confining the precursor in a spatially confined area can be achieved by physically pressing the wafers together to form a sandwich containing the precursor.

Suitable means of securing the assembly containing precursor in a spatially confined area include, without limitation, a scanning stage for moving the sample, a fixed sample mount and a scanning mirror system for moving the laser beam, and combinations thereof.

The assembly containing precursor in a spatially confined area can be raster scanned in two-dimensions normal to the incident laser beam such that the precursor layer remains located at the focus of the beam. The speed of the raster scan may be from about 1 μm/s to about 1 cm/s. The speed of the raster scan may be less than about 1 cm/s, less than about 1 mm/s, or less than about 10 μm/s. The speed of the raster scan may be more than about 1 μm/s, more than about 1 mm/s, or more than about 100 mm/s. Sample rastering can also include rotation of the sample specimen, for example rotation with respect to a line-focused laser light beam.

Suitable means for determining the presence of crystalline material on the surface of a polymer substrate include, without limitation, powder x-ray diffraction spectroscopy, scanning electron microscopy, transmission electron microscopy, Raman spectroscopy, and combinations thereof.

The various embodiments of the present invention could be used to modify a biological tissue by adding a crystalline material to the surface. For example, human skin could be modified with a variety of materials for potential cyborg applications. Plant tissue could be modified to be pest-resistant with various crystalline materials attached to the surface. All biological tissue could be modified to exist in environments that unmodified tissues could not survive.

Various features and advantages of the disclosure are set forth in the claims below. Although the disclosure above has been described in terms of various aspects and specific embodiments, it is not so limited. A variety of suitable alterations and modifications for operation under specific conditions will be apparent to those skilled in the art. It is therefore intended that the following claims be interpreted as covering all such alterations and modifications as fall within the spirit and scope of the invention.

All patents, publications and references cited herein are hereby fully incorporated by reference. In case of conflict between the present disclosure and incorporated patents, publications and references, the present disclosure should control.

### Examples

### Example 1

### Confined-Plume Chemical Deposition of Crystalline ReB₂ on Green Onion Tissue Using an 800-nm Laser

Green onion tissue (Allium chinense) and the crystalline ceramic material precursor (B₃H₈)Re(CO)₄ (prepared by the method of Gaines et al., “Syntheses and Properties of Some Neutral Octahydrotriborate(1-) Complexes of Chromium-, Manganese-, and Iron-Group Metals,” Inorg. Chem., 1978, 17, 794-806) were placed between two sapphire parallelepiped sheet wafers (available commercially from Thorlabs Inc., 56 Sparta Ave., Newton, N.J. 07860). The wafers were physically pressed together to form a wafer/tissue/precursor/wafer sandwich that spatially confined the onion cells and precursor in order to prevent vaporization of the precursor into the atmosphere and reaction of the precursor with atmospheric gases during the CPCD process.

Pulses of light were generated from an amplified Ti:sapphire laser (MIRA 900 oscillator from Coherent, Inc., 5100 Patrick Henry Dr., Santa Clara, Calif. 95054, and TITAN-1 amplifier from Quantronix, 41 Research Way, East Setauket, N.Y. 11733). The pulses of light had wavelength centered around about 800 nm, average energy per pulse of about 1 mJ, average pulse duration of about 150 fs FWHM, and a repetition rate of about 1 kHz. The pulses of light emerged from the laser in a collimated laser beam and were focused to a laser spot size of 0.150 mm×6 mm. The spatially confined green onion tissue and precursor of the precursor layer of the wafer/tissue/precursor/wafer sandwich were placed in the focus of the laser beam. A plasma and reaction plume were generated in the spatially confined area at the focus of the laser beam and ReB₂ crystals nucleated and grew on the surface of the onion cells. The wafer/tissue/precursor/wafer sandwich was raster scanned at 0.1 mm/s in two-dimensions normal to the incident laser beam such that the precursor layer remained located at the focus of the beam.

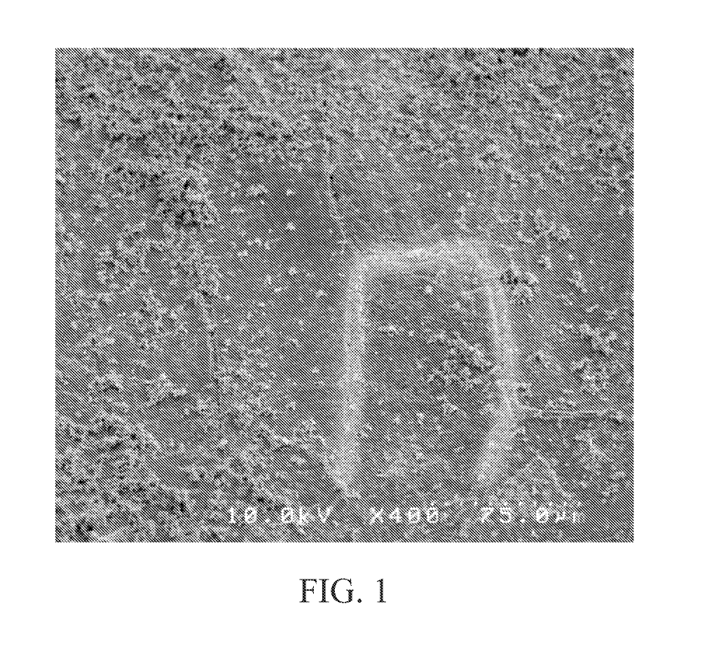

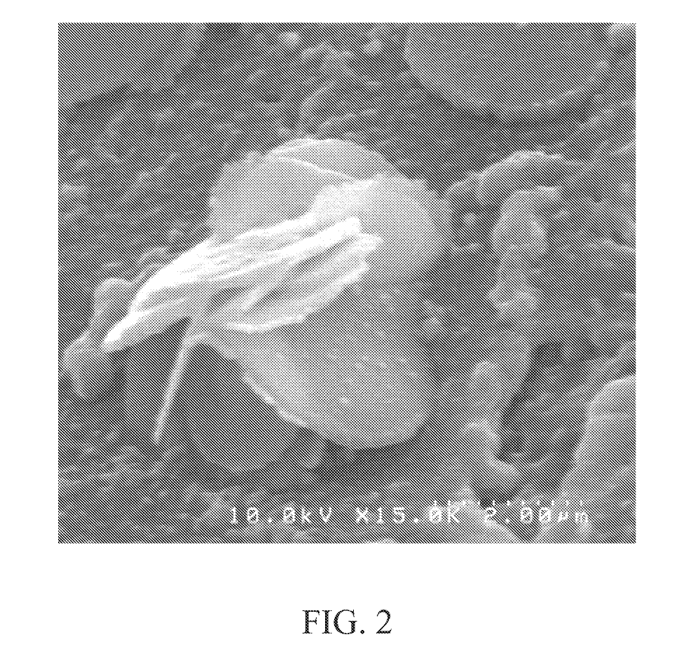

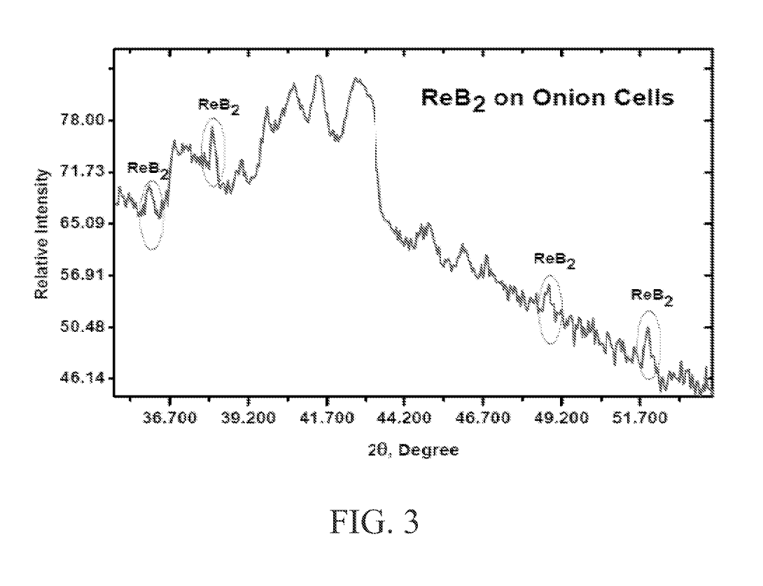

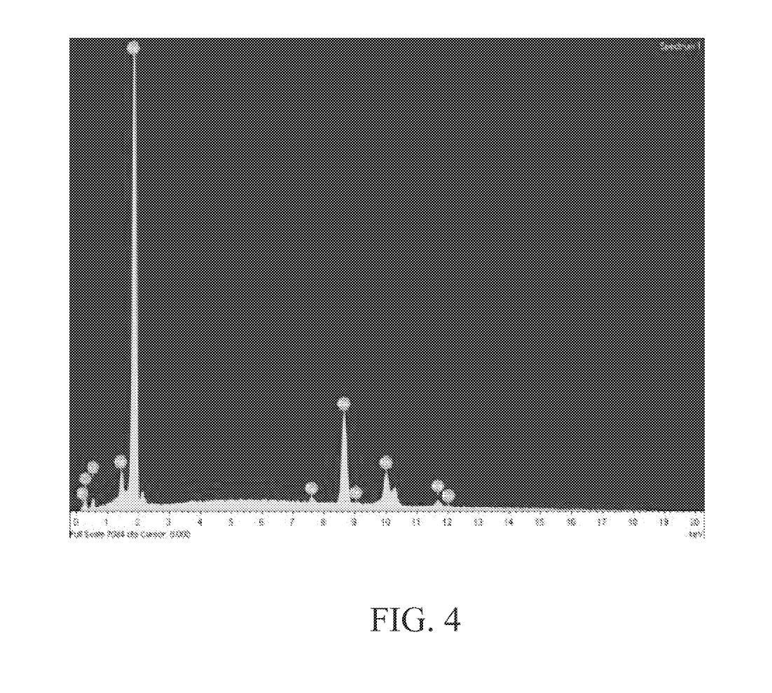

Formation of ReB₂ on the surface of onion cells was confirmed by scanning electron microscopy (Hitachi S-2400 scanning electron microscope with an accelerating voltage at either 5 or 20 kV), x-ray diffraction spectroscopy (Scintag X₁ theta/theta automated powder x-ray diffractometer with a Cu target, a Peltier-cooled solid-state detector and a zero-background Si(510) sample support), and energy-dispersive x-ray spectroscopy (Oxford EDX Model #6853 running at 20 keV), as shown in FIGS. 1-4.

### Example 2

### Confined-Plume Chemical Deposition of Crystalline CdS on Green Onion Tissue Using an 800-nm Laser

A circular wafer of green onion tissue (0.5 inch diameter×ca. 0.3 mm thickness) was coated with ca. 500 μg of Cd[S₂CN(CH₂CH₂CH₃)H]₂ (prepared by the method of L. H. van Poppel, T. L. Groy, and M. T. Caudle, Inorganic Chemistry, 43, 3180-3188 (2004)) and placed on a circular glass wafer (0.5 inch diameter×1 mm thickness). A circular sapphire wafer (0.5 inch diameter×ca. 3 mm thickness and available commercially from Thorlabs Inc., 56 Sparta Ave., Newton, N.J. 07860) was placed on top of the coated onion. The wafers were physically pressed together to form a glass wafer/tissue/precursor/sapphire wafer sandwich that spatially confined the green onion tissue and precursor in order to prevent vaporization of the precursor into the atmosphere and reaction of the precursor with atmospheric gases during the CPCD process.

Pulses of light were generated from an amplified Ti:sapphire laser (MIRA 900 oscillator from Coherent, Inc., 5100 Patrick Henry Dr., Santa Clara, Calif. 95054, and TITAN-1 amplifier from Quantronix, 41 Research Way, East Setauket, N.Y. 11733). The pulses of light had wavelength centered around about 800 nm, average energy per pulse of about 1 mJ, average pulse duration of about 150 fs FWHM, and a repetition rate of about 1 kHz. The pulses of light emerged from the laser in a collimated laser beam and were focused to a laser spot size of 0.1 mm×6 mm. The spatially confined green onion tissue and precursor of the precursor layer of the glass wafer/tissue/precursor/sapphire wafer sandwich were placed in the focus of the laser beam. A plasma and reaction plume were generated in the spatially confined area at the focus of the laser beam and CdS crystals nucleated and grew on the surface of the onion cells. The glass wafer/tissue/precursor/sapphire wafer sandwich was raster scanned at ca. 0.1 mm/s in two-dimensions normal to the incident laser beam such that the precursor layer remained located at the focus of the beam. The raster scanning was repeated four times.

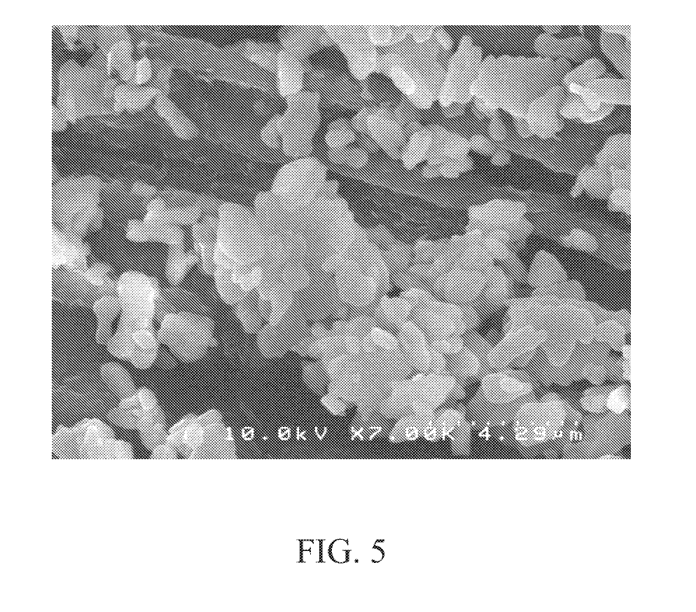

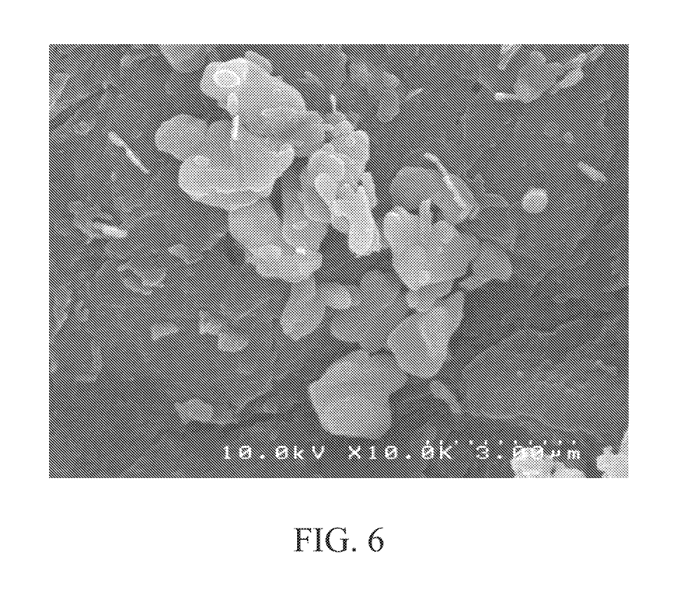

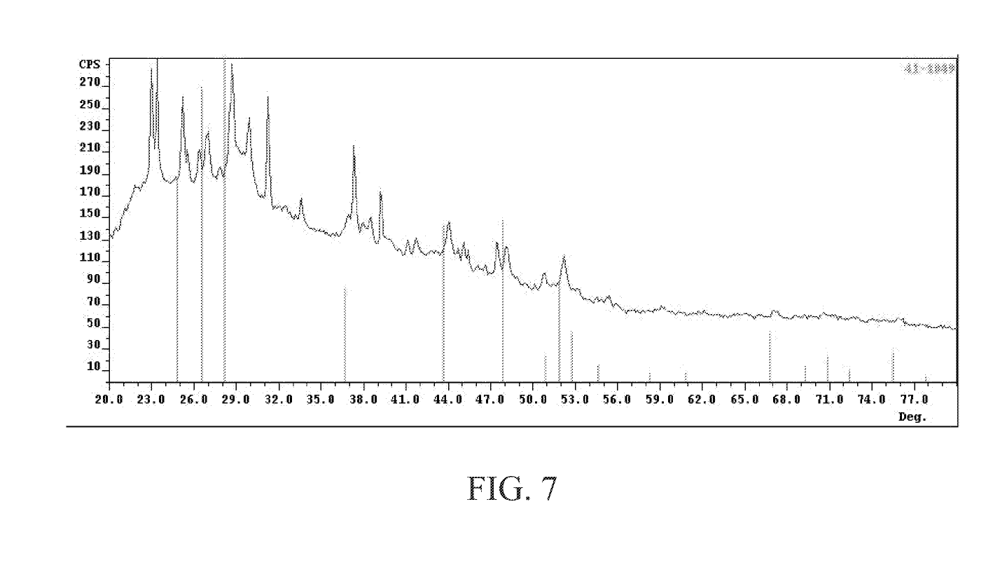

Formation of crystalline, wurtzite CdS platelets on the surface of green onion tissue was confirmed by scanning electron microscopy (Hitachi S-2400 scanning electron microscope with an accelerating voltage at either 5 or 20 kV) and x-ray diffraction spectroscopy (Scintag X₁ theta/theta automated powder x-ray diffractometer with a Cu target, a Peltier-cooled solid-state detector and a zero-background Si(510) sample support), as shown in FIGS. 5-7.

### Example 3

### Confined-Plume Chemical Deposition of Crystalline CdS on Green Onion Tissue Using a 2.94-μm Laser

The procedure of Example 2 was repeated with a different light source and scanning parameters.

Pulses of light were generated from a microsecond Er:YAG laser (CENTAURI Surgical Er:YAG Laser System by Premier Laser System, Inc., Irvine, Calif.). The pulses of light had wavelength centered around about 2.94 μm, average energy per pulse of about 150 mJ, average pulse duration of about 150 μs FWHM, and a repetition rate of about 10 Hz. The pulses of light emerged from the laser in a collimated laser beam and were focused to a laser spot size of 0.1 mm×6 mm. The spatially confined green onion tissue and precursor of the precursor layer of the glass wafer/tissue/precursor/sapphire wafer sandwich were placed in the focus of the laser beam. A plasma and reaction plume were generated in the spatially confined area at the focus of the laser beam and CdS crystals nucleated and grew on the surface of the onion cells. The glass wafer/tissue/precursor/sapphire wafer sandwich was raster scanned at ca. 0.4 mm/s in two-dimensions normal to the incident laser beam such that the precursor layer remained located at the focus of the beam. The raster scanning was repeated two times.

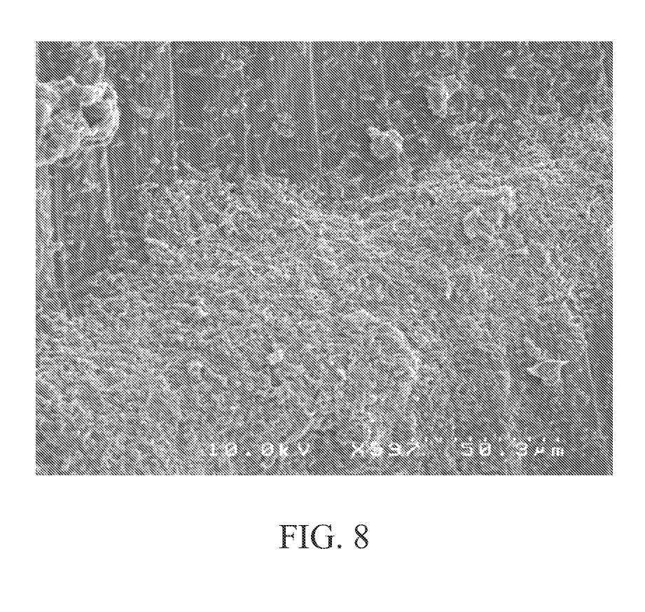

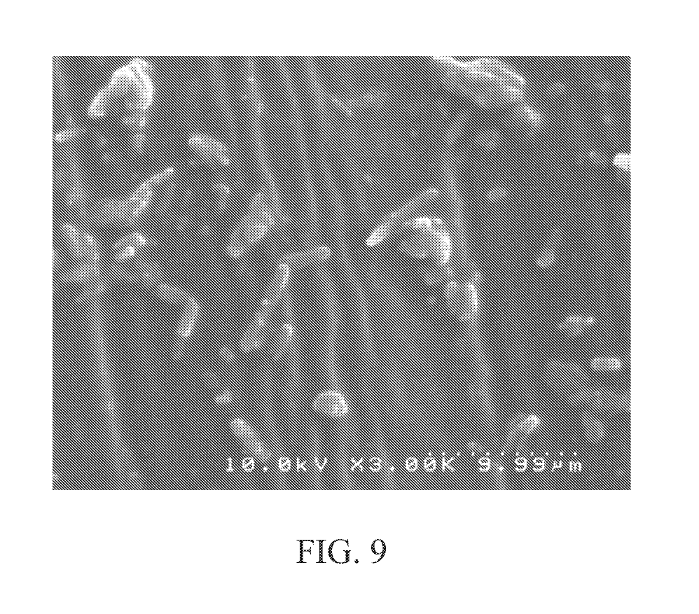

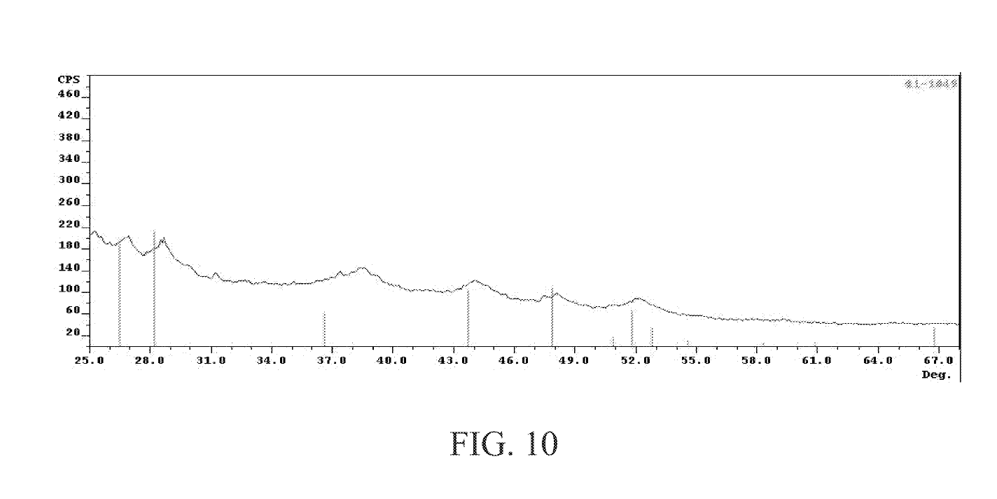

Formation of crystalline, wurtzite CdS rods on the surface of green onion tissue was confirmed by scanning electron microscopy (Hitachi S-2400 scanning electron microscope with an accelerating voltage at either 5 or 20 kV) and x-ray diffraction spectroscopy (Scintag X₁ theta/theta automated powder x-ray diffractometer with a Cu target, a Peltier-cooled solid-state detector and a zero-background Si(510) sample support), as shown in FIGS. 8-10.

### Example 4

### Confined-Plume Chemical Deposition of Crystalline Au on Green Onion Tissue Using an 800-nm Laser

The procedure of Example 2 was repeated replacing ca. 500 μg of Cd[S₂CN(CH₂CH₂CH₃)H]₂ with ca. 20 μL of a 0.16 M solution of bromo(triethylphosphine)gold (available commercially from Strem Chemicals Inc., 7 Mulliken Way, Newburyport, Mass. 01950) in chloroform, allowing the solvent to evaporate prior to placing the sapphire wafer on top of the coated onion, and replacing all instances of CdS with Au.

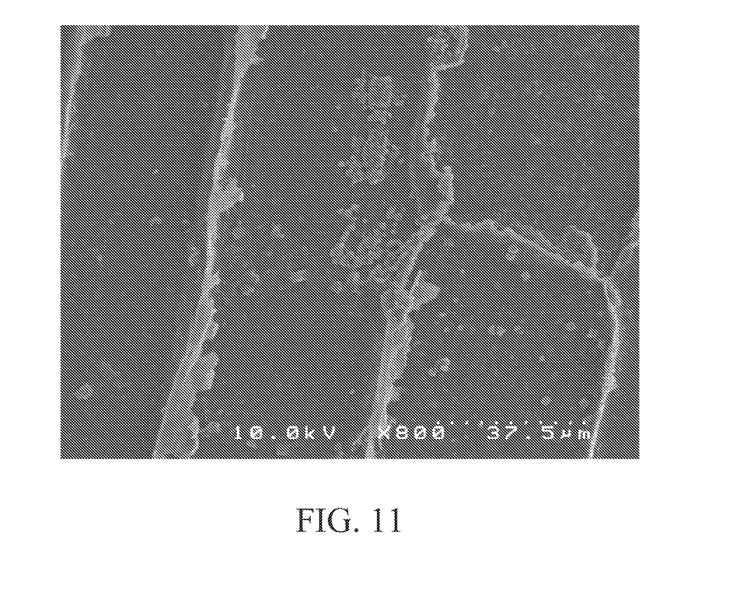

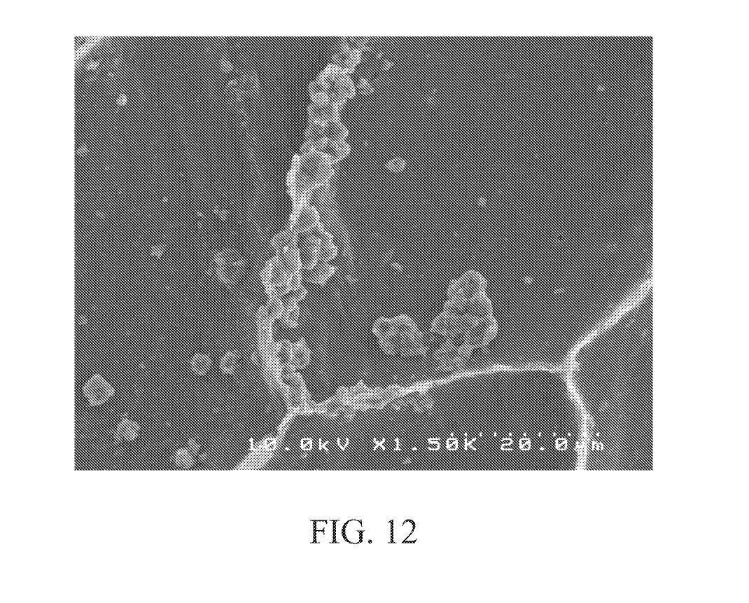

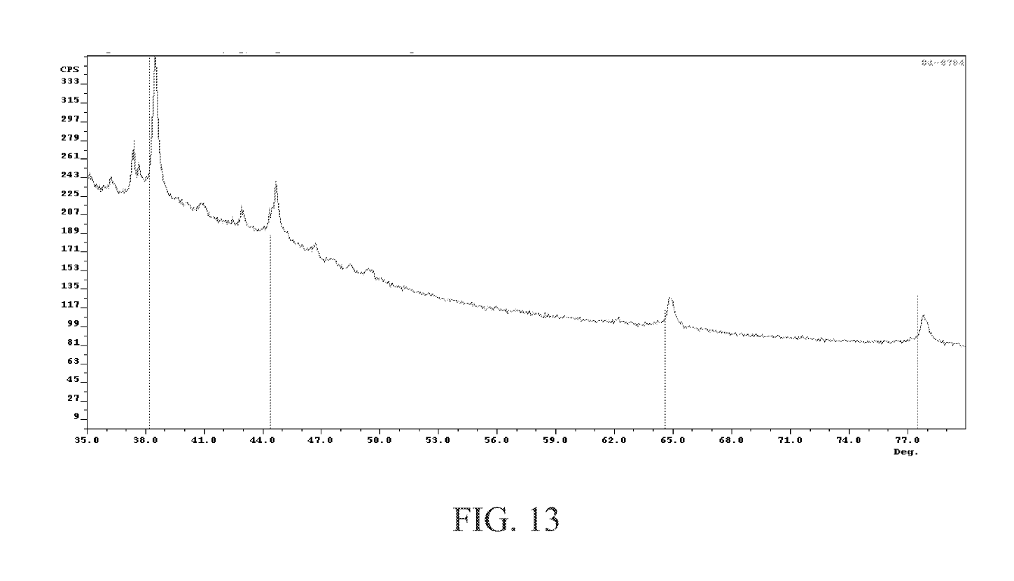

Formation of Au crystals on the surface of green onion tissue with average particle size of ca. 3 μm was confirmed by scanning electron microscopy (Hitachi S-2400 scanning electron microscope with an accelerating voltage at either 5 or 20 kV) and x-ray diffraction spectroscopy (Scintag X₁ theta/theta automated powder x-ray diffractometer with a Cu target, a Peltier-cooled solid-state detector and a zero-background Si(510) sample support), as shown in FIGS. 11-13.

### Example 5

### Confined-Plume Chemical Deposition of Crystalline ReB₂ on Green Onion Tissue Using an 800-nm Laser

The procedure of Example 1 was repeated, with the exception of raster scanning ten times rather than four time.

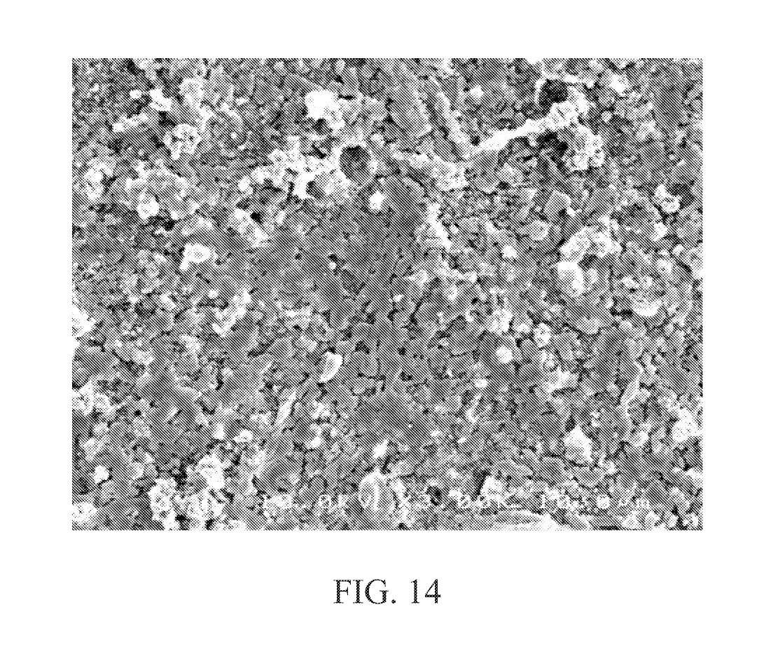

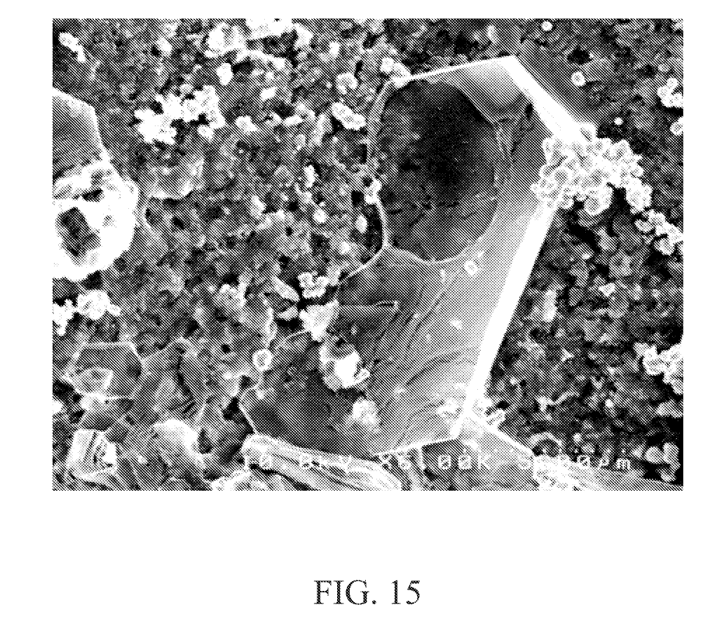

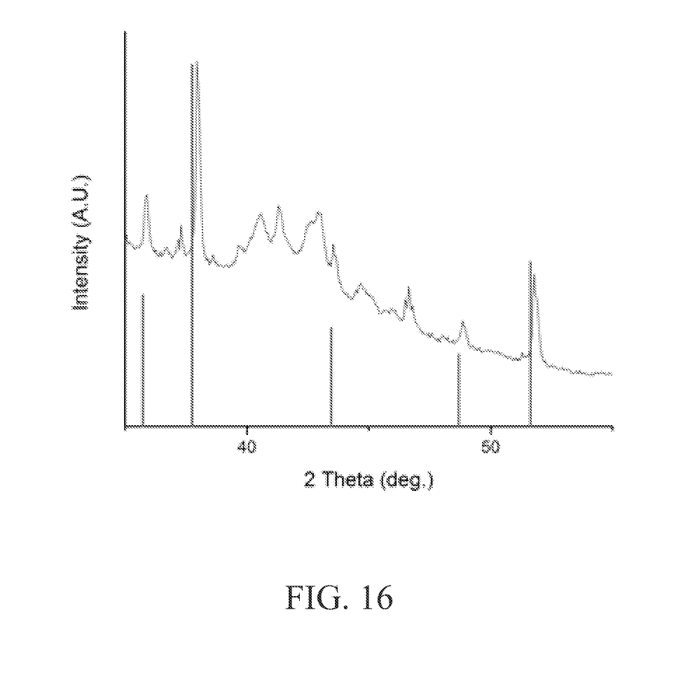

Formation of ReB₂ on the surface of onion cells was confirmed by scanning electron microscopy (Hitachi S-2400 scanning electron microscope with an accelerating voltage at either 5 or 20 kV) and x-ray diffraction spectroscopy (Scintag X₁ theta/theta automated powder x-ray diffractometer with a Cu target, a Peltier-cooled solid-state detector and a zero-background Si(510) sample support), as shown in FIGS. 14-16.

### Example 6

### Confined-Plume Chemical Deposition of Crystalline ReB₂ on Cow Femur Tissue Using an 800-nm Laser

A circular cross-section of domestic cow (Bos taurus) femur tissue was coated with ca. 50 μL of the crystalline ceramic material precursor (B₃H₈)Re(CO)₄ (prepared by the method of Gaines et al., “Syntheses and Properties of Some Neutral Octahydrotriborate(1-) Complexes of Chromium-, Manganese-, and Iron-Group Metals,” Inorg. Chem., 1978, 17, 794-806). A circular sapphire wafer (0.5 inch diameter×ca. 3 mm thickness and available commercially from Thorlabs Inc., 56 Sparta Ave., Newton, N.J. 07860) was placed on top of the coated cow femur tissue. The cow femur tissue and sapphire wafer were physically pressed together to form a tissue/precursor/sapphire wafer sandwich that spatially confined the precursor in order to prevent vaporization of the precursor into the atmosphere and reaction of the precursor with atmospheric gases during the CPCD process.

Pulses of light were generated second-harmonic generation (SHG) of a Nd:YLF laser (Quantronix Darwin Model 527DP-H Laser). The pulses of light had wavelength centered around about 527 nm, average energy per pulse of about 1 mJ, average pulse duration of about 100 nanosecond-200 nanosecond FWHM, and a repetition rate of about 1 kHz. The pulses of light emerged from the laser in a collimated laser beam and were focused to a laser spot size of 0.1 mm×6 mm. The spatially confined precursor of the cow femur tissue/precursor/sapphire wafer sandwich was placed in the focus of the laser beam, with the focus near the cow femur tissue. A plasma and reaction plume were generated in the spatially confined area at the focus of the laser beam and ReB₂ crystals nucleated and grew on the surface of the cow femur tissue. The cow femur tissue/precursor/sapphire wafer sandwich was raster scanned at ca. 0.1 mm/s in two-dimensions normal to the incident laser beam such that the precursor layer remained located at the focus of the beam. The raster scanning was repeated ten times.

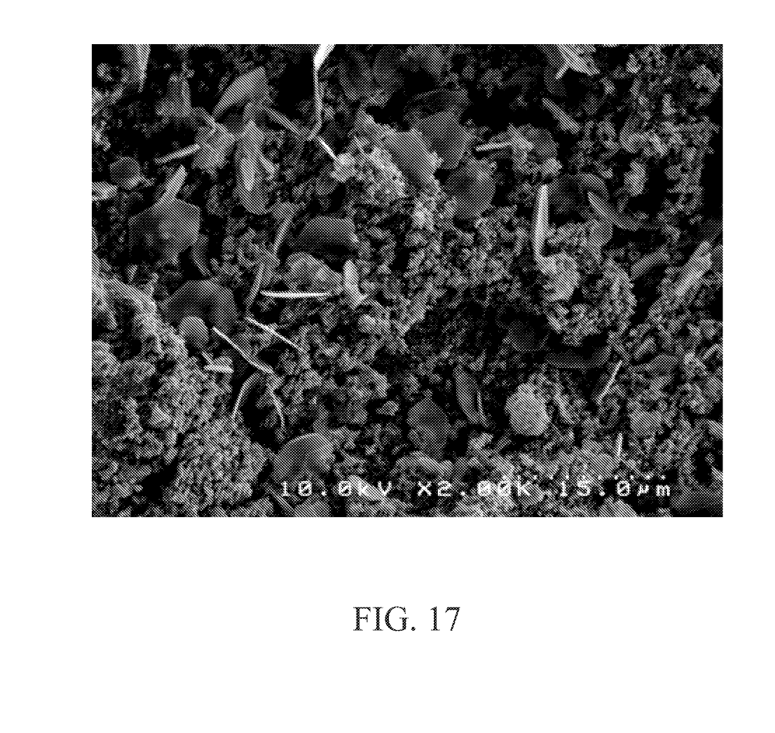

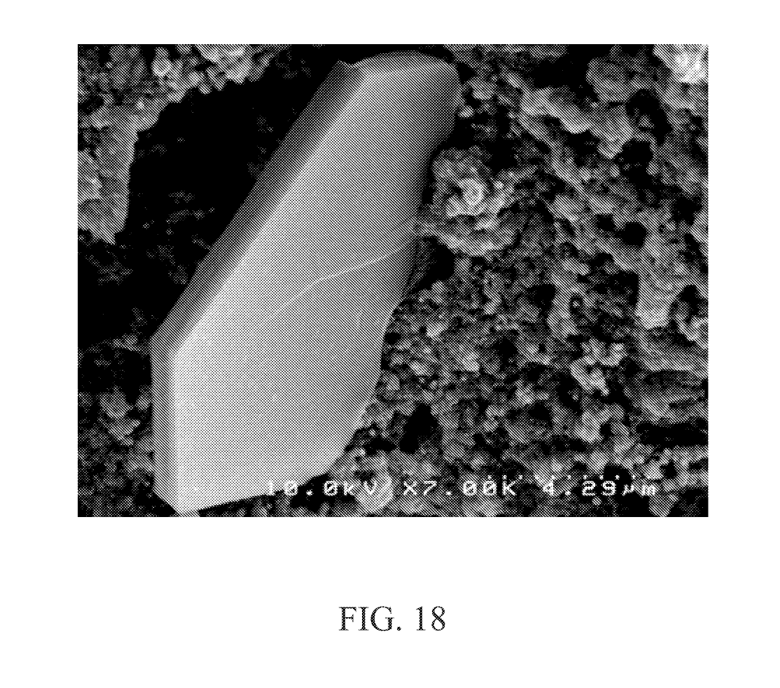

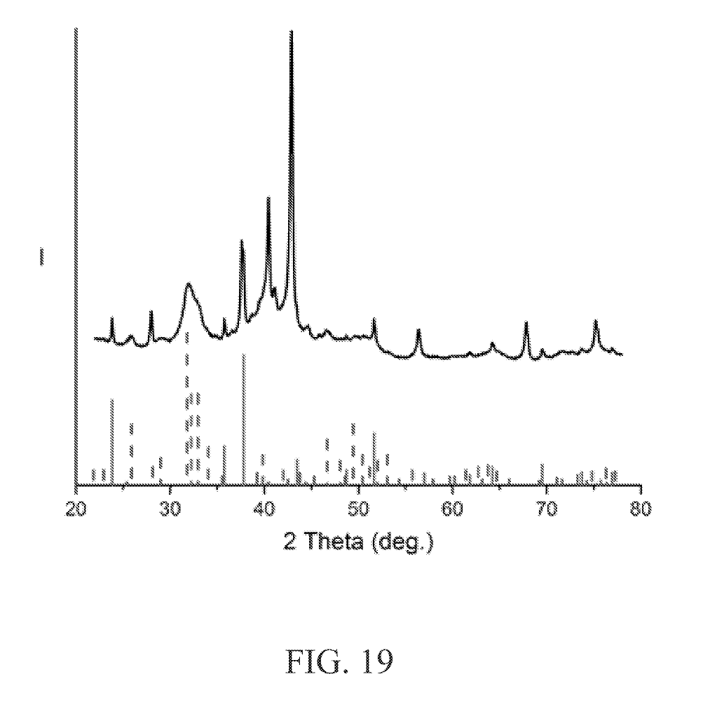

Formation of ReB₂ on the surface of cow femur tissue was confirmed by scanning electron microscopy (Hitachi S-2400 scanning electron microscope with an accelerating voltage at either 5 or 20 kV) and x-ray diffraction spectroscopy (Scintag X₁ theta/theta automated powder x-ray diffractometer with a Cu target, a Peltier-cooled solid-state detector and a zero-background Si(510) sample support), as shown in FIGS. 17-19.

What is claimed is:

1. (canceled)

2. (canceled)

3. A composition comprising a direct interface between a biological tissue and a crystalline material, wherein the crystalline material has a crystallization temperature that exceeds the temperature at which the biological tissue incurs thermal damage.

4. The composition of claim 3, wherein the biological tissue comprises plant or animal kingdom biological tissue.

5. (canceled)

6. The composition of claim 3, wherein the crystalline material comprises a crystalline ceramic material or a crystalline metal material.

7. The composition of claim 3, wherein the crystalline material comprises ReB₂.

8. (canceled)

9. The composition of claim 3, wherein the crystalline material comprises Au.

10. The composition of claim 3, wherein the crystalline material comprises a semiconductor material.

11. The composition of claim 3, wherein the crystalline material comprises CdS.

12. (canceled)

13. (canceled)

14. A method of forming a crystalline material directly on the surface of a biological tissue, the method comprising:
converting a material precursor into a crystalline material within a plasma generated by pulses of light in a spatially confined area; and
forming a modified biological tissue having the crystalline material thereon.

15. The method of claim 14, wherein the pulses of light have average duration less than about 1 μs (full width at half maximum).

16. (canceled)

17. The method of claim 14, wherein the pulses of light have wavelength centered from about 400 nm to about 1100 nm.

18. (canceled)

19. The method of claim 14, wherein the pulses of light have average duration less than about 500 μs (full width at half maximum).

20. The method of claim 14, wherein the pulses of light have average duration from about 1 μs (full width at half maximum) to about 300 μs (full width at half maximum).

21. The method of claim 14, wherein the pulses of light have wavelength centered from about 1 μm to about 10 μm.

22. The method of claim 14, wherein the pulses of light are produced by an Er:YAG laser or an amplified Ti:sapphire laser.

23. The method of claim 14, wherein the pulses of light have average energy from about 1 μJ to about 1 J.

24. The method of claim 14, wherein the pulses of light occur at a repetition rate from about 1 Hz to about 300 MHz.

25. The method of claim 14, wherein the pulses of light are focused to a spot size of less than about 1 cm×1 cm.

26. The method of claim 14, wherein the biological tissue comprises plant or animal kingdom biological tissue.

27. (canceled)

28. The method of claim 14, wherein the crystalline material comprises compounds selected from ReB₂, CdS and Au.

29. The method of claim 14, wherein the crystalline material precursor comprises a compound selected from (B₃H₈)Re(CO)₄, Cd[S₂CN(CH₂CH₂CH₃)H]₂, and bromo(triethylphospine)gold.

30. (canceled)

31. (canceled)

32. (canceled)

33. (canceled)
